(19) United States
(12) Patent Application Publication
(10) Pub. No.: US 2024/0252850 A1
(43) Pub. Date: Aug. 1, 2024

(54) WEARABLE DEVICE FOR THE TREATMENT OF BREATHING AIR

(71) Applicant: Gerardo D'AMBROSIO, Scarperia e San Piero (FI) (IT)

(72) Inventor: Gerardo D'AMBROSIO, Scarperia e San Piero (FI) (IT)

(21) Appl. No.: 18/560,976

(22) PCT Filed: May 30, 2022
(86) PCT No.: PCT/IB2022/055056

§ 371 Date: Nov. 15, 2023

(30) Foreign Application Priority Data
Jun. 4, 2021 (IT) 102021000014579

(51) Int. Cl. A62B 18/00; A61L 9/14; A61L 9/20; B05B 7/00; B05B 17/00; B05B 17/06

(52) U.S. Cl. CPC A62B 18/003; A61L 9/145; A61L 9/20; A62B 18/006; B05B 17/0646; A61L 2209/12; B05B 7/0012; B05B 17/0684

(57) ABSTRACT

The present invention relates to a device configured to be worn on the head of a user and provided with breathing air purification and treatment functionality. In particular, the invention relates to such a device in which the purification functionality is ensured by leaving the user's face substantially free.

### TECHNICAL FIELD OF THE INVENTION

The present invention relates to a device configured to be worn on the head of a user and provided with breathing air purification functionality. In particular, the invention relates to such a device in which the purification functionality is ensured by leaving the user's face substantially free.

### Background of the Invention

Devices of the type indicated above are known, which in a structure that can be said to be generically annular provide for conduits evolving at the sides or at the base of the user's head in such a way as to feed a flow of purified air towards the nose-buccal area of the user for inhalation by the user himself. To this end, the conduits are associated with, and mutually connected, both mechanically and pneumatically, to an air treatment unit adapted to be arranged in the rear region of the user's head and which comprises pneumatic flow propulsion means with impeller member, and air treatment means adapted to perform the function of purifying the pneumatic flow. Among the known solutions, some involve leaving the user's face substantially open and free, with the conduits terminating in openings that can be positioned at the sides of the aforesaid nose-buccal region. Among them, the devices shown for example in documents WO2015/140776, U.S. Ser. No. 10/821,255, CN106669056 are worth mentioning.

A solution of this type makes the device reasonably effective in ensuring a certain amount of protection from atmospheric pollution (making sure that the user can inhale a higher quantity of treated/filtered air than normal atmospheric air and/or of the external environment), while at the same time safeguarding the relational capacities ensured by the face left clear, and as well as obviously achieving a good wearing comfort, without discomfort that prevents the continuous or prolonged use thereof.

A functionality of suction and consequent treatment/filtering of the exhalation air may also be provided, but this tends to be provided in the context of devices that mask the nose-buccal area, such as the device that is the subject-matter of document WO2013/082650. Thus, in the latter case, these systems are hardly tolerable except in special circumstances and for limited periods of time.

In general, the forced ventilation system adopted in these known devices is in any case such as to create an open circuit, in which a substantial part of purified air is dissipated into the environment, and the filtration system has the burden of continuously producing new treated air starting from completely untreated ambient air.

In addition to this, the treatment and/or filtering unit adopted in such systems, although it may present acceptable performance in certain cases, is usually subject to periodic replacement operations of filters, cartridges or similar, or in any case force the user to frequent, laborious but also and above all very expensive maintenance operations. Some of the known devices are in any case relatively bulky, heavy and therefore not likely to encourage their use.

### SUMMARY OF THE INVENTION

Taking this state of the art into account, the Applicant has now developed a wearable breathing air purification device, of the type configured to be worn on the head, which, unlike to what is made available by the prior art, simultaneously achieves all of the following purposes:
high filtration or purification efficiency with reduced energy consumption and with a low-maintenance filtration/treatment system;
wearing comfort by leaving the nose-buccal area free, with consequent possibility of continuous use without compromising social and relational activity, and without the risks of hypoxia induced by mask systems of any kind;
efficacy both with respect to pollutants and with respect to infectious agents;
compactness, lightness and ergonomics.

These and other ancillary objects are achieved by the wearable breathing air treatment device, the essential characteristics of which are defined by the first of the appended claims. Other important additional features are the subject matter of the dependent claims.

### BRIEF DESCRIPTION OF THE FIGURES

The features and advantages of the wearable breathing air treatment device according to the present invention will become clearer from the following description of the embodiments thereof, made by way of example and not limitation, with reference to the accompanying drawings in which:

FIG. 1 is an axonometric view of a wearable device according to the present invention, in a first embodiment;

FIG. 2 shows schematically the device of FIG. 1 worn on the head of a user;

FIG. 3 is, again represented in axonometry, an exploded view of the device in the first embodiment of the previous figures;

FIG. 4 is a top view of the device of the previous figures, with parts split at different levels to highlight the pneumatic circuitry;

FIGS. 5 and 6 are sectional views of the device, with parts omitted of FIG. 6, according to section planes V-V and VI-VI of FIG. 4, respectively;

FIG. 7 is a cutaway axonometric view of an air treatment unit of the device in the previous figures;

FIG. 8 is an axonometric view of a wearable device according to the present invention, in a second embodiment;

FIG. 9 is, again represented in axonometry, an exploded view of the device in the second embodiment of FIG. 8;

FIG. 10 is a sectional view of the device of FIGS. 8 and 9, conducted on the prevailing development plane of the device; and

FIG. 11 is a sectional view of the device of the second embodiment, according to the section plane XI-XI of FIG. 10.

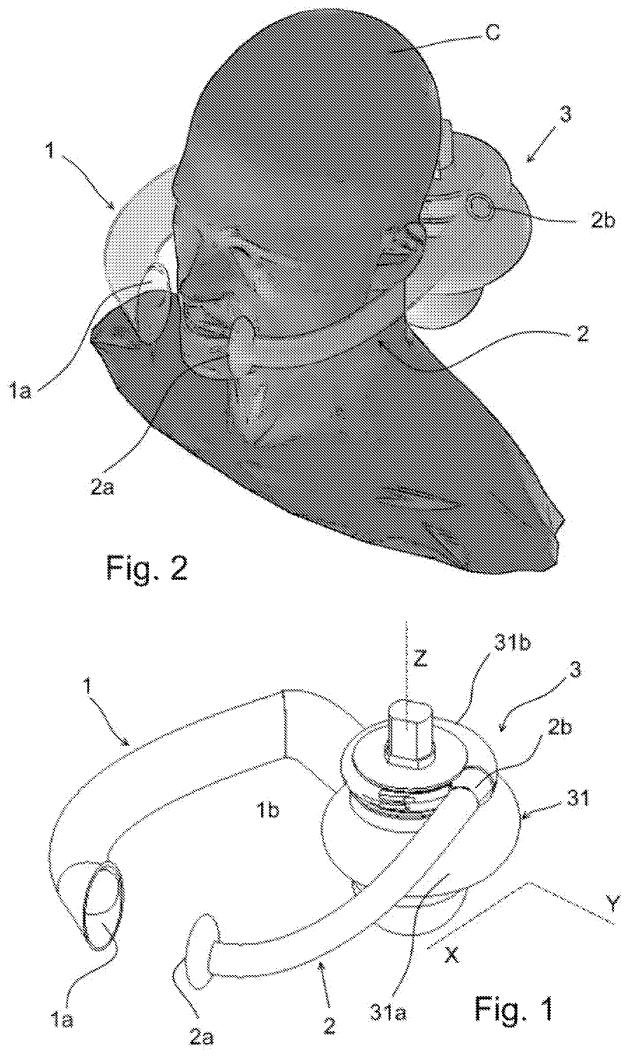

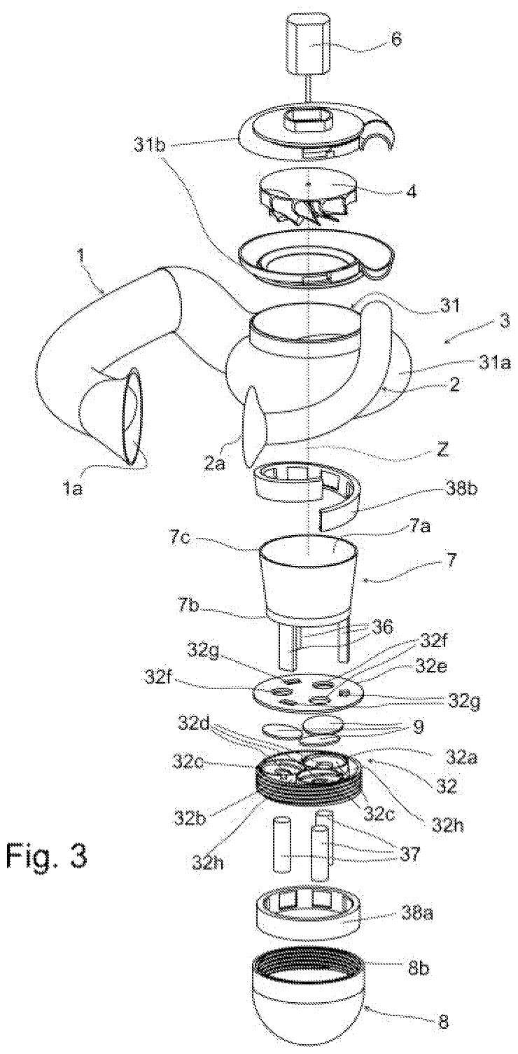

### DETAILED DESCRIPTION OF THE INVENTION

With reference to said figures, a device according to the invention has a substantially annular structure configured to be worn on the head C of a user (FIG. 2) while leaving the face substantially free.

The device comprises (numerical indices referring to both embodiments) at least one air suction conduit 1, 101, evolving between a suction inlet 1a, 101a of air to be treated containing suspended pollutant particles, which when the device is worn is arranged near the nose-buccal region of the user, and an air outlet 1b, 101b which is arranged in the near region of the user's head.

At least one ejection conduit 2, 102 then evolves between a treated air ejection outlet 2a, 102a, in turn arranged near the nose-buccal region (tendentially on the opposite side with respect to that where the suction inlet 1a, 101a faces) and in line with it, and an inlet 2b, 102b adapted to be arranged in the rear region of the head. Preferably, the section of the ejection conduit, at least at the end with the outlet gap, is smaller than the corresponding section (end with the suction inlet gap) of the suction conduit, in order to facilitate the priming of air.

The two conduits 1, 2, 101, 102 are mechanically and pneumatically (with the outlet of the suction conduit and with the inlet of the ejection conduit, respectively) joined to an air treatment unit 3, 103, which is therefore in turn adapted to be arranged in the rear region of the head.

The treatment unit 3, 103 comprises pneumatic flow propulsion means, for promoting the pneumatic flow from the suction conduit to the ejection conduit, practically realising a semi-closed loop circuit whereby air is sucked in from the naso-buccal region, passes through the treatment unit, and is ejected back into the aforesaid region. The only opening is represented by the volume of separation between the outlet-inlet of the respective conduits in the nose-buccal region of the user, a volume in which there is a prevalence of treated/healthy air available for the user's inspiration. However, due to the way in which the inlet-outlet gaps are positioned, it is a substantial part of the treated air emitted by the ejection conduit that is gradually sucked back by the suction conduit, and in this sense one can speak of a circuit approximating a semi-closed condition.

The treatment unit then comprises the actual treatment/filtering system 5, 105, which will be discussed in more detail shortly, and an energizing system such as a rechargeable battery-powered micromotor 6, 16 to move the impeller member and to energetically power the treatment/filtering system. The wirings, switches and in general what is generally required to ensure the operation of the simple electrical components adopted in the device are not represented, as they are of obvious nature and obviously implementable. For example, and in particular, obvious means will be provided for the regulation or partialisation of the flow rate and/or of the speed of the air flow within a pre-established intervention to adapt the device to its own needs, acting on the operation of the motor and/or on the section of the conduits.

Again according to the invention, the treatment/filtering system comprises a tubular chamber 7, 107, for example but not necessarily conical or more properly truncated conical, defining an inner lateral surface 7a, 107a, evolving according to an axis Z, Y between a first end 7b, 107b, having a smaller diameter and in pneumatic communication with the outlet of the suction conduit, and a second end 7c, 107c, having a larger diameter, in pneumatic communication with said inlet of the ejection conduit, and therefore influenced by the circulation promoted by the pneumatic flow propulsion means. Pneumatic flow directing means adapted to promote a swirling circulation within the chamber itself are also associated with the chamber.

According to a preferred solution, but which does not exclude other possible solutions, the propulsion means and the means for directing the pneumatic flow are made by the same component, an impeller member 4, 104 which is advantageously arranged coaxially to the chamber intercepting the inside thereof, with rotation axis Z, Y′ and configured with adequate blading like a centrifugal impeller.

A water reservoir 8, 108 communicates with the chamber, being placed near the first end 7b, 107b having a smaller diameter. Said reservoir is affected by water nebulizer means 9, 109, e.g. means for stirring the water contained therein, configured to promote an emission of nebulized water entering the chamber through the first end, whereby the nebulized water is invested by the air to be treated entering the chamber through the first end, and by effect of the swirling circulation promoted for example by the impeller, a deposit of water retaining said pollutant particles is formed in adhesion on the inner lateral surface of the chamber and tends to return to the reservoir, from which they can be disposed of periodically, with water supplementation/replacement, thanks to a reversible connection between the reservoir and the rest of the unit.

This being said in general terms, with more specific reference to the embodiment options, and therefore here to the first embodiment and to the relative Figures from 1 to 7, the treatment unit 3 is configured with the axis Z of rotation of the impeller and of development of the chamber that is arranged orthogonally to the plane XY of the general development of the device, i.e. a plane defined by the layout of the conduits and which in the arrangement of use of the device corresponds to a transverse plane of the head. In practice, in this arrangement of use and with the user in an upright position, the axis Z is substantially vertical.

The treatment unit 3 in this case comprises a box-like body 31 for housing and supporting the other components, defined by a lower portion 31a and by an upper portion 31b which are generically disc-shaped, having a larger cross-section for the lower portion, which evolve with polar symmetry with respect to the axis Z. The suction 1 and ejection 2 conduits engage respectively in the lower portion and in the upper portion of the body with a substantially tangential angle.

The lower portion or volute 31a is shut at the base by a pervious diaphragm 32, lying orthogonally to the axis Z and to which we will return later, which thus separates the inside of the box-like body 31 from the underlying reservoir 8, components among which a reversible connection can advantageously, as mentioned, operate, for example with screws 32c-8b with suitable seals (not shown). The lower portion 31a also centrally houses a sleeve 7, for example just conical, which materializes the above-mentioned chamber, so that the second end 7c, in this case upper, is adjacent to the upper portion or volute 31b. Around the sleeve 7 the lower portion 31a develops a lower toroidal channel 33 for distributing air, fed by the suction conduit 1, around the base of the sleeve itself. The latter with the first end 7b thereof is spaced with respect to the diaphragm 32, so as to create a passage 34 of the pneumatic flow between the toroidal channel and the inside of the sleeve or chamber 7.

The upper portion 31b centrally houses the centrifugal impeller 4, which as mentioned is arranged at the second end 7c of the conical sleeve 7, defining around it an upper toroidal channel 35, for the radial distribution of the air and for directing it towards the ejection conduit 2.

Returning to the pervious diaphragm 32, in the illustrated embodiment it has a main septum 32a with a distribution of seats 32c housing respective stirring elements in the form of porous piezoelectric foils 9 with which the aforesaid nebulizer means are advantageously made. In this embodiment solution, the water is fed to the foils, which by vibrating at an appropriate frequency take charge of stirring, and consequent diffusion of nebulized water within the chamber 7 from the first end 7b, advantageously exploiting the phenomenon of capillarity through ducts 37 which rise from the inside of the reservoir and penetrate the septum 32a suitably provided with holes 32d, below the seats 32c. These ducts are, for example, materialized by bodies of known structure, made of porous or spongy material such as cellulose acetate, which form small channels precisely suitable to produce the physical phenomenon of the rise of water by capillarity. Beyond this possible embodiment solution by vibration, which, as mentioned, is advantageous in terms of simplicity and efficiency, the nebulizer means may include other types of expedients adapted to produce an equivalent result in the formation of an aerosol.

The diaphragm 32 is completed by a cover 32e superimposed on the septum 32a and which closes the seats 32c of the piezoelectric foils, bearing at their correspondence a first distribution of holes 32f to allow the passage of nebulized water towards the chamber. The plate-shaped cover 32e also bears a second distribution of holes 32g, which correspond to passages 32h of the septum 32a, within which respective discharge tubes 36 penetrate and extend from the first end 7b of the conical sleeve 7, the first end being folded inwards to form a perimeter shower 7d which has the function of collecting the water mixed with impurities falling along the inner lateral surface 7a, a water that the communication between the shower and the discharge tubes 36 allows to precipitate back into the reservoir.

The device can still be provided with other treatment systems, such as one or more UVC-radiating LED strips, in this case a lower annular strip 38a in the reservoir 8 and an upper helical strip 38b around the sleeve 7, all to add precisely a germicidal action that as we know can be carried out by the aforesaid type of radiation, suitably adjusted.

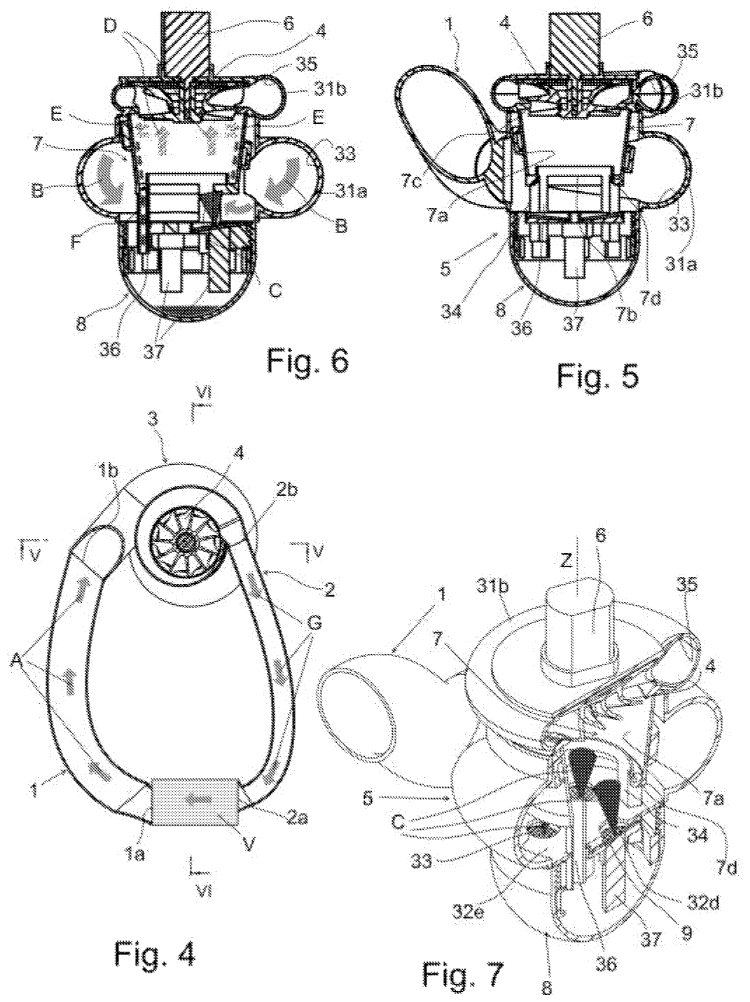

The device described so far therefore operates as follows, with particular reference to FIGS. 4 and 6 in which the arrows schematize the air and water flows. Once worn on the head and the conduits are in place, the outlet gap 2a of the ejection conduit 2 and the inlet gap 1a of the suction conduit 1 are located in the naso-buccal region, delimiting an exchange volume V which is influenced by the pneumatic action of the device and which represents precisely an air exchange volume, as it faces the breathing cavities, invested by the user's inhalation and exhalation activity.

During inspiration, the incoming flow taken from the volume V is replenished by air coming from the surrounding environment. During the exhalation phase, the breath joins the flow entering the device. In this way, the device repeatedly processes and purifies a large mass of air, comprising the same exhaled air, increasing the degree of cleanliness.

Following the path of the air to be treated, which comprises pollutants in the form of particulate matter, ash, volatile substances in suspension, etc., which path as said is promoted by the impeller 4, the air then enters the suction conduit 1 through the inlet or suction inlet gap 1a (arrows A of FIG. 4) and reaches the lower toroidal channel 33 of the lower portion, distributing itself at the base of the sleeve 7 and being drawn axially into it through the first end 7b thereof (arrows B of FIG. 6). Here the air invests the emission of nebulized water that rises from the diaphragm 32 due to the vibration of the piezoelectric foils 9 (schematized by the conical regions C of FIG. 6 and FIG. 7). The action of the vortex induced by the impeller by suction on the mixture of air and polluting particles/dust favours the aggregation of these particles in suspension with the micro-droplets of water produced by the foils. This mixture is drawn towards the impeller within the chamber defined by the sleeve and along the axis thereof by a swirling circulation (the axial component of which is represented by the arrows D of FIG. 6).

By approaching the second end 7c of the chamber 7, the swirling flow takes on a strongly centrifugal component (arrows E of the same FIG. 6). The tangential speed course of the mixture grows as it approaches the impeller mouth, consequently increasing the centrifugal effect. Near the mouth of the impeller 4, the droplets and the incorporated material thus tend to settle on the inner lateral surface 7a, while the air enters the compartments of the impeller blading.

Due to the diverging diameter shape of the surface, and to the pressure gradient generated in the chamber, this substantially liquid deposit tends to descend towards the first end 7b and from there by gathering in the shower 7d and through the discharge tubes 36 (arrows F of FIG. 6) is deposited at the bottom of the reservoir 8 where the polluting component removed from the pneumatic flow also accumulates. Impurities gathered in the reservoir, where they settle at the bottom, can be removed by unscrewing the reservoir, an operation with which consumed water will also be gradually replenished. The treated air is distributed downstream of the impeller into the upper toroidal channel 35 and from it into the ejection conduit 2 (arrows G of FIG. 4) to join again the exchange volume V outside the device, laterally investing the mouth and the nose of the user, who will benefit from the inhalation of air that is for the most part treated and purified.

The device thus ensures a continuous inflow of air, which eliminates all problems related to hypoxia phenomena induced by traditional filter masks. The continuous circular flow allows large amounts of air to be (re)treated several times (the percentage of air already treated can be as high as 85-90% of the air being gradually sucked in), thus allowing to increase the level of air purification. The configuration of the device allows the front of the face to be left unobstructed, guaranteeing an absolute benefit in relational/social terms. By appropriately sizing the impeller and the passage sections of the pneumatic circuit, it will be possible to ensure a redundant volume supply of emitted and treated air (up to 60 litres air/min) and a high flow speed (e.g. around 10 m/s), which requirements ensure the highest quality of the air that the user inhales.

Appropriate aromatic or even medicinal substances can be dissolved in the water reservoir, which once nebulized are breathed in by the user, bringing further benefits. More generally speaking, it should be pointed out that, when mentioning water, in the context of the present disclosure, it was meant to indicate the simplest liquid that tends to be apt for use, without, however, ruling out the possibility that other liquid substances may be used to the extent that they are suitable for ensuring the operation of the device.

Evidently, the device does not require filters that need being replaced periodically, once they have reached their saturation. The use of water (preferably distilled) to operate the filtration has no environmental impact and requires simply replenishing it after a certain number of hours, depending on the conditions of use.

The conduits are made wholly or partially of material or technology that is suitable to provide them with adequate resilience, possibly even with a certain elasticity, so as to ensure on the one hand the deformability necessary for wearing, and on the other hand adaptability to the physiognomy of the user and contextually a wearing that is stable and at the same time comfortable. However, it is not excluded that the device can be equipped with accessories or aids to further increase comfort and stability, e.g. padding, head support laces and the like. Likewise, the conceptual configuration of the device could be integrated into more wraparound structures (helmets or similar).

The construction of the treatment unit can easily be made with dismountable components, to facilitate washing with running water and suitable detergents to remove any impurities that may remain in the conduits, before subsequent use. The adoption of UVC LED technology enhances sterilisation functionalities to combat microorganisms and viruses that are potentially dangerous to human health.

The power supply battery for the supply of adequate autonomy to the motor, to the nebulizer means and to any UVC LED means can be rechargeable according to standard technologies, for example by means of a USB connector. The motor can be a simple 6V micromotor adapted to generate, for example, an impeller rotation speed of 12/13000 rpm. Depending on the embodiment, other technologies or speed ranges may be more appropriate.

The person skilled in the art will also have no difficulty in implementing any sensors capable of monitoring in real time the quality of the air treated, the reservoir filling, and other control parameters of the device. These sensors may eventually dialogue, by equipping the device with appropriate electronic and communication components, with a dedicated application developed for smartphones, so as to manage the device via the application itself, as well as monitor personal parameters relating, for example, to respiratory coefficients. The sharing of certain collected data via network, subject to authorisation by users, may also allow real-time air quality mapping to be carried out in a defined region.

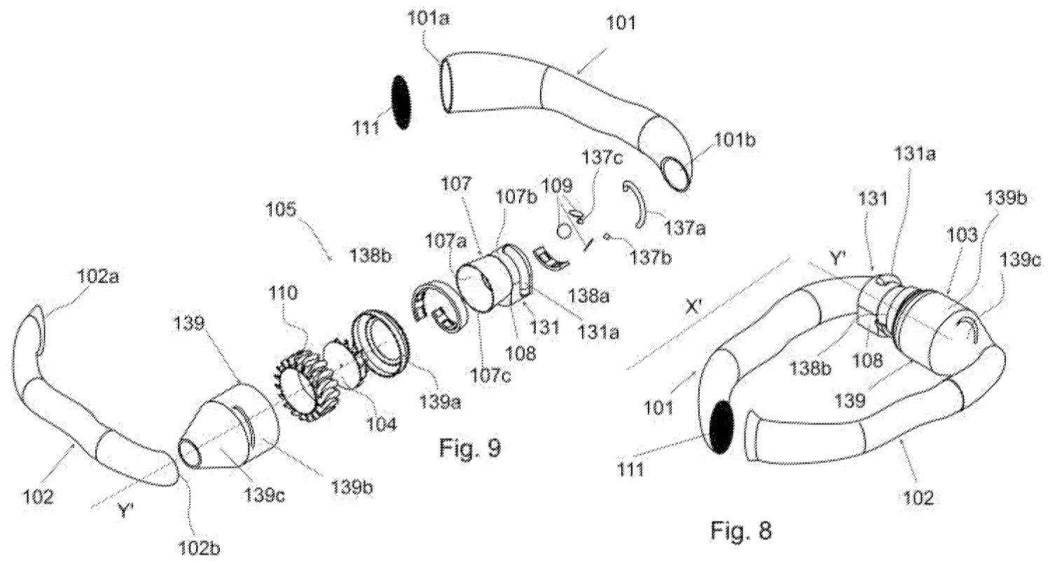

Above and beyond what has been described for the first embodiment, it is clear that the invention can be reduced to practice with different construction solutions. Among these, in a second embodiment, which is the specific subject of FIGS. 8 to 11 to which reference is made below, and in the treatment unit 103 the common axis of the impeller and of the chamber, indicated in this case with Y′, is lying on the plane X′Y′ of general development of the device as already defined above, or in any case parallel to it. In practice, in the arrangement of use and with the user in an upright position, the axis Y′ is substantially horizontal and oriented in the direction of laterality.

The first end having a smaller diameter 107b and the second end having a larger diameter 107c of the chamber 107 (defining the inner surface 107a) are thus oriented, again with the device worn, on the respective sides of the user's head, and the suction 101 and ejection 102 conduits are joined there with a substantially (co)axial angle. Consequently, the pneumatic flow develops with the main component remaining fundamentally axial.

This different configuration gives rise to a number of different construction options of this embodiment, described below remaining in the context of a construction that envisages a centrifugal impeller member to promote and direct the flow (although this construction expedient is not in principle the only one possible also for the embodiment now considered).

With regard to the liquid stirring/feeding functionality, in this case, between the outlet 101b of the suction channel and the first end 107b of the chamber 107, it may be provided for a cylindrical tubular manifold 131 for entering the chamber, having an internal diameter substantially corresponding to that of the chamber 107 at the first end 107b. On the inner surface of the manifold 131 there are defined seats 132 which house respective piezoelectric foils 109, to which water is fed by capillary ducts 137a, 137b, 137c which extend between respective seats 132 and a reservoir 108 herein configured as a radial expansion of the manifold in the lower area (i.e. which is arranged below when the device is worn). To this end, the ducts in this case have different developments, a first duct 137a feeding liquid to the foil furthest from the reservoir having in particular an elongated and arched development according to the circumference of the manifold on which an appropriate rib 131a is provided for the purpose, and two other ducts 137b, 137c for conveyance to foils closest to the reservoir and with substantially radial development.

The lower radial expansion, which as mentioned forms the reservoir 108, can advantageously have an alveolus 108a at the bottom in which the collection of impurities is favoured.

Turning to the outlet area of the purified pneumatic flow, an outlet manifold 139 from the chamber is arranged between the second end 107c of the chamber and the inlet 102b of the ejection conduit 102, and comprises, in succession following the axial course of the pneumatic flow a cylindrical segment 139b immediately downstream of the chamber, with a diameter corresponding to that of the latter at the second end, and a conical segment 139c which narrows the passage section up to that of the ejection conduit.

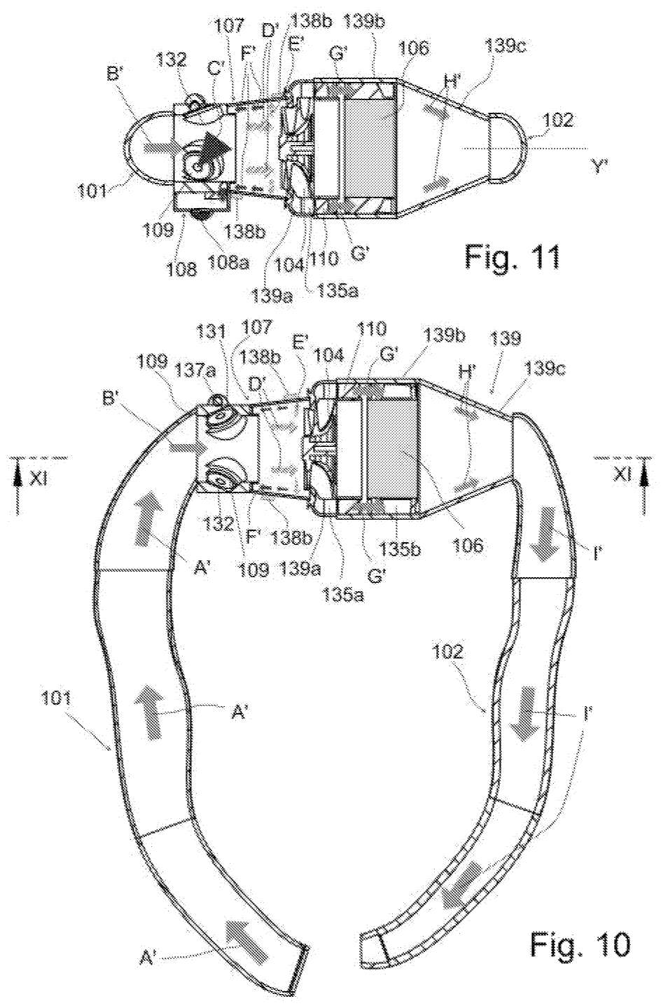

More specifically, the cylindrical segment 139b is joined to the chamber 107 by means of a connecting cup 139c in which the centrifugal impeller 104 is centrally housed and which peripherally defines a toroidal channel 135a for distributing and directing the outgoing air. Downstream of the impeller, and thus in the cylindrical segment 139b as such, there is a rotor 110 driven in rotation together with the impeller 104 and evidently coaxially with it. The rotor 110 is configured with an axial blading to further promote the flow from the toroidal channel 135a in an axial direction, and to inject it into an annular cavity 135b, which again within the cylindrical segment 139b surrounds a housing compartment for the motor 106 (schematically shown in FIGS. 10 and 11 only). A stator element, not represented here, is generally useful for conveying the circulation in the axial direction between impeller and rotor, according to what is obvious to the person skilled in the art.

Finally, the conical segment 139c has the task of conveying the pneumatic flow up to the inlet 102b of the ejection conduit 102. UVC-radiating LED strips are or can be provided in this embodiment as well, in the form of a first arc strip 138a in the reservoir 8 and an upper helical strip 138b around sleeve 107. Finally, the drawings of this embodiment exemplify an evolution of the conduits which envisages, as already provided above in general and evidently also usable in the context of the first embodiment, at least resilient, deformable or articulated central sections to improve adaptation to the user's head, and a mesh filter 111 which can intercept the inlet 101a of the suction conduit is also represented.

Following also in this case the path of the air to be treated, it then enters the suction conduit 101 arrows A′ of FIG. 10) and arrives directly in an axial direction at the base of the sleeve 107, being drawn axially into it through the first end 107b thereof (arrows B′ of FIG. 10 and FIG. 11). Here, the air invests the water nebulized by the piezoelectric foils 109 (conical region C′ in FIG. 11). Again, it is the vorticity induced by the impeller that favours the aggregation of the suspended particles with the micro-droplets of water produced by the foils. This mixture is drawn towards the impeller within the chamber and along the axis thereof by a swirling circulation (the axial component of which is represented by the arrows D′ of FIG. 11).

Towards the second end 107c of the chamber 107, the centrifugal component (arrows E′) of the flow pushes the droplets and the incorporated material so as to settle on the inner lateral surface 107a, while air enters the compartments of the impeller. It is mainly the pressure gradient generated in the diverging chamber that in this case promotes drawing the liquid deposit towards the first end 107b and the accumulation in the reservoir 108 (arrows F′).

The treated air is distributed downstream of the impeller in the toroidal channel 135a and from it through the blading of the axial rotor 110 into the cavity 135b of the cylindrical segment of the manifold 139, again with axial direction indicated by the arrows G′, and is then conveyed into the conical segment 139c (arrows H′) and then enters the ejection conduit 102 (arrows I′).

This second embodiment also evidently does not exhaust the construction options according to which the present invention can be practically implemented. As much it has been described so far with reference to preferred embodiments, it is therefore to be understood that other embodiments may exist within the scope of protection of the attached claims.

1. A wearable air treatment device having a generically annular structure configured to be worn on the head of a user while leaving the face of the user substantially unobstructed, the device comprising:
an air suction conduit, evolving between a suction inlet of air to be treated containing suspended pollutant particles, adapted to be arranged near a nose-buccal region of the user, and an outlet of air adapted to be arranged in a rear region of the user's head;
an air ejection conduit evolving between a treated air ejection outlet adapted to be arranged near the nose-buccal region of the user and an inlet adapted to be arranged in the rear region of the user's head;
an air treatment unit connected to said conduits and in pneumatic communication therewith between said outlet of said suction conduit and said inlet of said ejection conduit, said unit being adapted to be arranged in the rear region of the user's head and comprising: pneumatic flow propulsion means from said suction conduit to said ejection conduit; air treatment means configured to receive air to be treated from said outlet of said suction conduit, and return treated air to said inlet of said ejection conduit; and energization means of said pneumatic flow propulsion means and said air treatment means;
wherein said air treatment means comprise:
a tubular chamber defining an inner side surface of the chamber and evolving along an axis of the chamber between a first end, having a smaller diameter and in pneumatic communication with said outlet of the suction conduit, and a second end, having a larger diameter, in pneumatic communication with said inlet of said ejection conduit;
pneumatic flow directing means configured to promote a swirling circulation within said chamber;
a water reservoir communicating with said tubular chamber and placed near said first end; and
nebulizer means associated with said reservoir, configured to promote an emission of nebulized water entering said chamber through said first end, whereby:
said emission of nebulized water is invested by the air to be treated entering said chamber from said first end, and by effect of said swirling circulation a deposit of water retaining said pollutant particles is formed in adhesion on said inner lateral surface of said chamber and tends to return to said reservoir.

2. The device according to claim 1, wherein said pneumatic flow propulsion means comprise an impeller member.

3. The device according to claim 2, wherein said pneumatic flow directing means are formed by said impeller member, configured as a centrifugal impeller and coaxially supported within said tubular chamber.

4. The device according to claim 1, wherein said chamber has a truncated conical shape.

5. The device according to claim 1, wherein said nebulizer means comprise water agitator means in said reservoir.

6. The device according to claim 5, wherein said water agitator means comprise one or more piezoelectric elements.

7. The device according to claim 6, wherein said one or more piezoelectric elements comprise one or more foils made of porous material, said nebulizer means further comprising capillary ducts extending between said reservoir and said foils to supply water from said reservoir to said foils.

8. The device according to claim 7, wherein said capillary ducts are provided by one or more bodies made of porous or spongy material.

9. The device according to claim 1, further comprising one or more UVC radiation LEDs adapted to perform a germicidal action on water in said reservoir.

10. The device according to claim 6, further comprising a first LED strip arranged at the level of said reservoir and a second helical LED strip evolving around said chamber.

11. The device according to claim 1, wherein said conduits comprise one or more resilient, deformable or articulated sections to improve adaptation to the head of the user.

12. The device according to claim 1, further comprising a suction conduit and an ejection conduit lying substantially over a plane of general development of the device, which in the use position of the device corresponds to a transverse plane of the user's head.

13. The device according to claim 12, wherein said chamber is arranged with said chamber axis orthogonal to said general development plane of the device.

14. The device according to claim 13, wherein said treatment unit comprises a box-like housing body defined by a lower portion and an upper portion, both generically disc-shaped and evolving with substantially polar symmetry with respect to said chamber axis, said suction and ejection conduits joining said body respectively at said lower portion and at said upper portion according to a substantially tangential angle, said chamber being formed by a sleeve housed in said lower portion so that said second end of the chamber is adjacent to said upper portion, said lower portion being shut at its base by a pervious diaphragm supporting said nebulizer means, said diaphragm separating the inside of said body from said reservoir, said lower portion forming a lower toroidal channel for distributing air, fed by the suction conduit, around the base of the sleeve and towards the inside of the chamber through a passage between the first end of the chamber and said diaphragm, said upper portion supporting said energizing means, centrally housing said impeller member and forming around it an upper toroidal channel for the radial distribution of the air and feeding it towards the ejection conduit.

15. The device according to claim 14, wherein said pervious diaphragm provides a distribution of seats for accommodating said one or more piezoelectric foils, and a distribution of holes for said capillary ducts and for discharge tubes extending from the first end of the sleeve, said first end of the sleeve being folded inwards to form a peripheral gutter adapted to collect water mixed with impurities falling along the inner lateral surface of the chamber.

16. The device according to claim 12, wherein said chamber is arranged with said chamber axis parallel to, or lying on, said general development plane of the device.

17. The device according to claim 16, further comprising a chamber intake tubular manifold between said outlet of the suction channel and said first end of the chamber, said manifold having an inner diameter substantially corresponding to that of the chamber at the first end, on the inner surface of the manifold seats being formed for housing respective piezoelectric foils, to which water is fed by said capillary ducts extending between respective seats and said reservoir, the latter being configured as a radial expansion of the manifold at a lower zone.

18. The device according to claim 17, wherein said radial expansion forming said reservoir has at its bottom a cavity assisting the collection of the pollutant particles.

19. The device according to claim 16, wherein a chamber outtake manifold is arranged between the second end of the chamber and said inlet of the ejection conduit, and comprises, in succession following the axial course of the pneumatic flow, a cylindrical segment immediately downstream of the chamber, having a diameter corresponding to that of said second end of the chamber, and a conical segment which narrows the air passage section to that of the ejection conduit, said cylindrical segment being joined to the chamber by means of a connecting cup in which said centrifugal impeller member is housed and which peripherally defines a toroidal channel for distributing and directing the outgoing air, downstream of said impeller member, and therefore towards the cylindrical segment, in the latter a rotor being arranged, driven into rotation together with said impeller member and configured with an axial vane to further promote the flow from said toroidal channel in the axial direction, and to introduce it into an annular gap which still inside the cylindrical segment surrounds a housing for said energizing means.

20. The device according to claim 1, wherein said reservoir is supported detachably from the rest of the device.

21. The device according to claim 1, wherein said energizing means comprise a motor and rechargeable battery means.
